(19) United States
(12) Patent Application Publication
(10) Pub. No.: US 2012/0264953 A1
(43) Pub. Date: Oct. 18, 2012

(54) ZINC-COMPRISING CATALYST FOR PREPARING ETHYLENE OXIDE

(75) Inventors: Tobias Rosendahl, Mannheim (DE); Torsten Mäurer, Ludwigshafen (DE); Cornelia Katharina Dobner, Ludwigshafen (DE); Jürgen Zühlke, Speyer (DE)

(73) Assignee: BASF SE, Ludwigshafen (DE)

(21) Appl. No.: 13/446,475

(22) Filed: Apr. 13, 2012

Related U.S. Application Data
(60) Provisional application No. 61/475,242, filed on Apr. 14, 2011.

(51) Int. Cl. C07D 301/10; B01J 37/08; B01J 27/02; B01J 37/02; B01J 23/66; B01J 21/04

(52) U.S. Cl. 549/534; 502/343; 502/342; 502/219

(57) ABSTRACT

Process for preparing a catalyst for preparing ethylene oxide, which comprises silver and zinc applied to a support, where the process comprises at least
(i) providing a support,
(ii) applying zinc to the support according to (i) in an amount of from 10 ppm to 1500 ppm, in each case calculated as element and based on the total weight of the support according to (i), by bringing the support into contact with at least one mixture G1 comprising at least one zinc compound,
(iii) drying and/or calcining the support obtained according to (ii),
(iv) applying silver to the dried and/or calcined support by bringing the support into contact with at least one mixture G2 comprising at least one silver compound.

The present invention relates to a process for preparing a catalyst for preparing ethylene oxide, which comprises silver and zinc applied to a support, and also a catalyst which can be or has been produced by this process. In addition, the present invention relates to a process for preparing ethylene oxide from ethylene, which comprises the oxidation of ethylene in the presence of such a catalyst.

Ethylene oxide is an important basic chemical and in industry is frequently prepared by direct oxidation of ethylene by means of oxygen in the presence of silver-comprising catalysts. Supported catalysts to which the catalytically active, metallic silver has been applied by means of a suitable process are frequently used. As support material, it is in principle possible to use various porous materials such as activated carbon, titania, zirconia or silica or ceramic compositions or mixtures of these materials. In general, alpha-alumina is used as support. Examples of disclosures concerning the direct oxidation of ethylene are DE-A-2300512, DE-A 2521906, EP-A-0014457, DE-A-2454972, EP-A-0172565, EP-A-0357293, EP-A-0266015, EP-A-0011356, EP-A-0085237, DE-C2-2560684 and DE-A-2753359.

Apart from silver as active component, these catalysts often comprise promoters to improve the catalytic properties. Examples of promoters are alkali metal compounds and/or alkaline earth metal compounds. Some documents teach the use of transition metals such as tungsten or molybdenum. A particularly preferred promoter for influencing the activity and/or selectivity of catalysts is rhenium. Catalysts comprising rhenium and/or other transition metal promoters in combination with alkali metal compounds and/or alkaline earth metal compounds are preferably used in industry because of their high selectivity. For the purposes of the present invention, the selectivity is the molar percentage of ethylene which reacts to form ethylene oxide. The activity is characterized by the ethylene oxide concentration at the reactor outlet at otherwise constant conditions such as temperature, pressure, amount of gas, amount of catalyst, etc. The higher the ethylene oxide concentration in the outlet stream from the reactor, the higher the activity of the catalyst. The lower the temperature required to achieve a particular alkylene oxide concentration, the higher the activity.

To achieve a high selectivity, the combination of the alkali metals and the composition of the support have to be matched precisely to one another in order to obtain catalysts having very good properties.

Thus, for example, U.S. Pat. No. 4,410,453 describes a catalyst composed of silver applied to a zinc-comprising support, with zinc being used in the form of zinc oxide as support additive in the preparation of the support. Here, the addition of from about 4 to 30% by weight of zinc, calculated as oxide, and based on the total weight of the support, to the raw material for the support is disclosed. This addition leads, under the calcination conditions used, to the formation of zinc spinel. The catalyst comprises neither rhenium nor other promoters. The catalysts formed in this way display selectivities in the range from 60 to 70%.

U.S. Pat. No. 4,007,135 likewise describes a catalyst comprising silver applied to a support, with metal promoters, including zinc, being applied together with the silver to the support.

Proceeding from this prior art, it was an object of the present invention to provide a process for preparing novel catalysts for the epoxidation of ethylene and also novel catalysts which can be or have been produced by this process and have advantageous activities and/or selectivities.

According to the invention, this object is achieved by a process for preparing a catalyst, which comprises silver and zinc applied to a support, and a catalyst which has been or can be produced by this process, where the process comprises at least
(i) providing a support,
(ii) applying zinc to the support according to (i) in an amount of from 10 ppm to 1500 ppm, in each case calculated as element and based on the total weight of the support according to (i), by bringing the support into contact with at least one mixture G1 comprising at least one zinc compound,
(iii) drying and/or calcining the support obtained according to (ii),
(iv) applying silver to the dried and/or calcined support by bringing the support into contact with at least one mixture G2 comprising at least one silver compound.

Furthermore, the invention describes a catalyst for preparing ethylene oxide itself, where the catalyst comprises a support and silver and zinc applied to this support and zinc has been applied in an amount of from 10 ppm to 1500 ppm, in each case calculated as element and based on the total weight of the support, to the support.

It has surprisingly been found that zinc-comprising catalysts in which from 10 ppm to 1500 ppm of zinc have been applied to the support and in which the zinc has not been applied together with silver but instead in a separate step, in particular a preceding step, to the support display particularly advantageous properties. In particular, the catalysts display advantageous initial selectivities and/or selectivities in the preparation of ethylene oxide. For the purposes of the present invention, the term “initial selectivity” is the molar percentage of ethylene which is converted into ethylene oxide during the first 80 hours of operation.

### Step (i)

Examples of suitable materials for the support provided in step (i) of the process of the invention are alumina, silica, silicon carbide, titania, zirconia and mixtures thereof, with alumina being preferred.

In a preferred embodiment, the present invention accordingly provides a process as described above or a catalyst which can be or has been produced by this process, where the support is an alumina support. Furthermore, the present invention also describes a catalyst as described above itself, where the support is an alumina support.

The term alumina as used in the present context comprises all conceivable structures such as alpha-, gamma- or theta-alumina. In a preferred embodiment, the support is an alpha-alumina support.

In a further preferred embodiment, the alpha-alumina has a purity of at least 75%, preferably a purity of at least 80%, more preferably a purity of at least 85%, more preferably a purity of at least 90%. For example, the alpha-alumina has a purity of at least 98%, at least 98.5% or at least 99%.

The term alpha-alumina accordingly also comprises alpha-aluminas comprising further constituents, in particular constituents selected from the group consisting of zirconium, alkali metals, alkaline earth metals, silicon, zinc, gallium, hafnium, boron, fluorine, copper, nickel, manganese, iron, cerium, titanium, chromium and mixtures of two or more thereof.

The alpha-alumina can comprise the constituents in any suitable form, for example as element or in the form of one or more compounds. If the alpha-alumina comprises one or more constituents in the form of a compound, it comprises this as, for example, oxide or mixed oxide.

As regards the amount of the further constituents, the total content of the further constituents is preferably in the range of less than 25% by weight, more preferably less than 20% by weight, more preferably less than 15% by weight and more preferably less than 10% by weight, based on the total weight of the support and calculated as sum of the elements other than aluminum and oxygen.

If the support comprises, for example, silicon, it preferably comprises this in an amount in the range from 50 to 10000 ppm, more preferably in an amount of from 100 to 5000 ppm, more preferably in an amount of from 1000 to 2800 ppm, based on the total weight of the support and calculated as element.

In a particularly preferred embodiment of the invention, the support is an alpha-alumina support, where the alpha-alumina has a purity of at least 85% and comprises silicon in an amount in the range from 50 to 10000 ppm, more preferably in an amount of from 100 to 5000 ppm and more preferably in an amount of from 1000 to 2800 ppm.

If the support comprises, for example, alkali metals, it preferably comprises these in a total amount in the range of not more than 2500 ppm, more preferably in a total amount of from 10 to 1500 ppm, more preferably in a total amount of from 50 to 1000 ppm, based on the total weight of the support and calculated as element.

In a preferred embodiment, the support comprises at least one alkali metal, in particular sodium and/or potassium.

The present invention therefore also provides a process as described above and a catalyst which can be or has been produced by this process, where the support comprises at least one alkali metal, in particular sodium and/or potassium. Furthermore, the present invention also describes a catalyst as described above itself, where the support comprises at least one alkali metal, in particular sodium and/or potassium.

If the support comprises sodium, it preferably comprises this in an amount in the range from 10 to 1500 ppm, more preferably in an amount of from 10 to 1000 ppm, more preferably in an amount of from 10 to 800 ppm, more preferably in an amount of from 10 to 500 ppm, based on the total weight of the support and calculated as element, with the total amount of all alkali metals as described above preferably being in the range from 10 to 2500 ppm.

If the support comprises potassium, it preferably comprises this in an amount of not more than 1000 ppm, more preferably in an amount of not more than 500 ppm, more preferably in an amount of not more than 200 ppm, for example in the range from 10 to 200 ppm, based on the total weight of the support and calculated as element, with the total amount of all alkali metals as described above preferably being in the range from 10 to 2500 ppm.

In a preferred embodiment of the invention, the support comprises sodium in an amount of from 10 to 1500 ppm and potassium in an amount of not more than 1000 ppm.

In a further embodiment, the support comprises at least one alkaline earth metal. If the support comprises at least one alkaline earth metal, it preferably additionally comprises at least one alkali metal as described above.

If the support comprises at least one alkaline earth metal, it preferably comprises a total amount of alkaline earth metals in the range of not more than 2500 ppm, for example in the range from 1 to 2500 ppm, more preferably in an amount of from 10 to 1200 ppm, more preferably in an amount of from 100 to 800 ppm, based on the total weight of the support and calculated as element. The expression “total amount of alkaline earth metals” as used here relates to the sum of all alkaline earth metals optionally comprised in the support, based on the total weight of the support and calculated as element.

In an embodiment of the invention, the support comprises at least one alkaline earth metal selected from the group consisting of calcium and magnesium.

If the support comprises, for example, calcium, it preferably comprises this in an amount in the range from 10 to 1500 ppm, more preferably in an amount of from 20 to 1000 ppm, more preferably in an amount of from 30 to 500 ppm, based on the total weight of the support and calculated as element.

If the support comprises, for example, magnesium, it preferably comprises this in an amount in the range of not more than 800 ppm, preferably in an amount of from 1 to 500 ppm, more preferably in an amount of from 1 to 250 ppm, more preferably in an amount of from 1 to 150 ppm, based on the total weight of the support and calculated as element.

The present invention therefore also provides a process as described above for preparing a catalyst and a catalyst which can be produced by this process as described above, where the support comprises magnesium in an amount of not more than 800 ppm and calcium in an amount of from 10 to 1500 ppm, in each case based on the total weight of the support and calculated as element. The present invention likewise provides a catalyst as described above itself, where the support comprises magnesium in an amount of not more than 800 ppm and calcium in an amount of from 10 to 1500 ppm, in each case based on the total weight of the support and calculated as element.

The support particularly preferably comprises sodium in an amount of from 10 to 1500 ppm, potassium in an amount of not more than 1000 ppm, magnesium in an amount of not more than 800 ppm and calcium in an amount of from 10 to 1500 ppm, in each case based on the total weight of the support and calculated as element.

For the purposes of the invention, particular preference is given to the support comprising zirconium If the support comprises zirconium, it preferably comprises zirconium in an amount in the range from 1 to 10000 ppm, more preferably in the range from 10 to 8000 ppm, more preferably in the range from 50 to 6000 ppm and particularly preferably in the range from 50 to 5000 ppm, calculated as metal and based on the total weight of the support.

In addition to the zinc which is applied to the support in step (ii), the support provided according to (i) itself, in particular the alpha-alumina support, can comprise zinc as further constituent. If the support comprises zinc as further constituent, it comprises this in an amount of not more than 1000 ppm, preferably in an amount of not more than 250 ppm, more preferably in an amount in the range from 1 to 250 ppm, calculated as element and based on the total weight of the support.

The present invention therefore also provides a process as described above and a catalyst which can be or has been produced by this process, wherein the support provided according to (i) comprises from 1 to 1000 ppm of zinc and from 1 to 10000 ppm of zirconium, in each case calculated as element and based on the total weight of the support.

In an alternative preferred embodiment, the support provided according to (i) does not comprise any zinc.

If the support comprises further constituents selected from the group consisting of gallium, hafnium, boron, fluorine, copper, nickel, manganese, iron, cerium, titanium and chromium, it preferably comprises each of these in an amount of not more than 500 ppm, in each case calculated as metal and based on the total weight of the support.

The supports used according to the invention preferably comprise a BET surface area determined in accordance with DIN ISO 9277 of from 0.1 to 5 m²/g, more preferably in the range from 0.1 to 2 m²/g, more preferably in the range from 0.5 to 1.5 m²/g, more preferably in the range from 0.7 to 1.3 m²/g, more preferably in the range from 0.7 to 1.2 m²/g and particularly preferably in the range from 0.7 to 1.0 m²/g.

The present invention therefore also provides a process as described above for preparing a catalyst and a catalyst which can be produced by this process, wherein the support has a BET surface area determined in accordance with DIN ISO 9277 in the range from 0.1 to 5 m²/g, more preferably in the range from 0.1 to 2 m²/g, more preferably in the range from 0.5 to 1.5 m²/g, more preferably in the range from 0.7 to 1.3 m²/g, more preferably in the range from 0.7 to 1.3 m²/g, more preferably in the range from 0.7 to 1.2 m²/g and particularly preferably in the range from 0.7 to 1.0 m²/g. The present invention likewise provides a catalyst as described above itself, wherein the support has a BET surface area determined in accordance with DIN ISO 9277 in the range from 0.1 to 5 m²/g, more preferably in the range from 0.1 to 2 m²/g, more preferably in the range from 0.5 to 1.5 m²/g, more preferably in the range from 0.7 to 1.3 m²/g, more preferably in the range from 0.7 to 1.3 m²/g, more preferably in the range from 0.7 to 1.2 m²/g and particularly preferably in the range from 0.7 to 1.0 m²/g.

Furthermore, the supports according to the invention preferably have pores having diameters in the range from 0.1 to 100 μm, with the pore distribution being able to be monomodal or polymodal, for example bimodal, trimodal or tetramodal. The supports preferably have a bimodal pore distribution. The supports more preferably have a bimodal pore distribution having peak maxima in the range from 0.1 to 10 μm and from 15 to 100 μm, preferably in the range from 0.1 to 5 μm and from 17 to 80 μm, more preferably in the range from 0.1 to 3 μm and from 20 to 50 μm, more preferably in the range from 0.1 to 1.5 μm and from 20 to 40 μm. The pore diameters are determined by Hg porosimetry (DIN 66133). The expression “bimodal pore distribution having peak maxima in the range from 0.1 to 10 μm and from 15 to 100 μm” as used above means that one of the two peak maxima is in the range from 0.1 to 10 μm and the other peak maximum is in the range from 15 to 100 μm.

The present invention therefore also provides a process as described above for preparing a catalyst and a catalyst which can be produced by this process, wherein the support has a polymodal pore distribution, preferably a bimodal pore distribution, more preferably a bimodal pore distribution comprising at least pores having diameters in the range from 0.1 to 10 μm and pores having pore diameters in the range from 15 to 100 μm, determined by Hg porosimetry in accordance with DIN 66133. The present invention likewise provides a catalyst as described above itself, wherein the support has a polymodal pore distribution, preferably a bimodal pore distribution, more preferably a bimodal pore distribution at least comprising pores having pore diameters in the range from 0.1 to 10 μm and pores having pore diameters in the range from 15 to 100 μm, determined by Hg porosimetry in accordance with DIN 66133.

The geometric shape of the supports is generally of minor importance, but the supports should advantageously be present in the form of particles which allow unhindered diffusion of the reaction gases to a very large part of the catalytically active external and internal surface area of the support which is coated with silver particles and optionally coated with further promoters. In addition, a very low pressure drop over the total reactor length has to be ensured by the chosen geometric shape of the support.

In a preferred embodiment, the support is used as shaped body, for example as extrudate, hollow extrudate, star extrudate, sphere, ring or hollow ring. The catalyst is preferably used in the form of a hollow ring. The shaped bodies usually have a volume equivalent diameter of from about 2.5 to 12 mm, preferably from about 3 to 10 mm. The term “volume equivalent diameter” indicates the diameter of a sphere having the same volume as the shaped body under consideration.

The support is preferably a shaped body having the geometry of a hollow body. Particular preference is given to cylinders having the following geometries (external diameter×length×internal diameter, in each case indicated in mm): 5×5×2, 6×6×3, 7×7×3, 8×8×3, 8×8.5×3, 8×8.5×3.5, 8.5×8×3.5, 8.5×8×3, 9×9×3, 9.5×9×3, 9.5×9×3.5. Each length indicated comprises tolerances in the range ±0.5 mm.

The water absorption of the supports is preferably in the range from 0.35 ml/g to 0.65 ml/g, preferably in the range from 0.42 ml/g to 0.52 ml/g, determined by vacuum cold water uptake.

In general, a suitable catalyst support for the present invention can be produced by mixing the alumina with water or another suitable liquid, with a burn-out material or a pore former and at least one binder. Suitable pore formers are, for example, cellulose and cellulose derivatives, e.g. methylcellulose, ethylcellulose, carboxymethylcellulose, or polyolefins such as polyethylenes and polypropylenes, or natural burn-out materials such as ground walnut shells.

The pore formers are selected so that they are burned out completely at the selected furnace temperatures in the calcination of the alumina to give the finished alpha-alumina support. It is important that they are burned out completely since this critically influences the porosity and the shape of the pores. Suitable binders and extrusion aids are described, for example, in EP 0 496 386 B2. Mention may be made by way of example of alumina gels with nitric acid or acetic acid, cellulose, e.g. methylcellulose, ethylcellulose or carboxyethylcellulose, or methyl or ethyl stearate, polyolefin oxides, waxes and the like.

The paste formed by mixing can be brought to the desired shape by extrusion. Extrusion aids can be used to assist the extrusion process.

After shaping, the shaped body obtained as described above is usually optionally dried and calcined to give the alumina support according to (i). Calcination is usually carried out at temperatures in the range from 1200° C. to 1600° C. It is usual to wash the alumina support in an appropriate way after calcination in order to remove soluble constituents.

Suitable supports can be obtained, for example, from N or Pro Co.

### Step (ii)

In step (ii), zinc is applied in an amount of from 10 ppm to 1500 ppm, in each case calculated as element and based on the total weight of the above-described support, to the support according to (i) by bringing the support into contact with at least one mixture G1 comprising at least one zinc compound.

The mixture G1 preferably comprises the at least one zinc compound as a solution in at least one solvent, in particular as a solution in water. The at least one zinc compound is preferably selected from the group consisting of zinc chloride, zinc nitrate, zinc acetate, zinc bromide, zinc iodide, zinc carbonate, zinc hydroxide, zinc oxalate and zinc acetylacetonate. The mixture G1 can further comprise a complexing agent such as ethanolamine, EDTA, 1,3- or 1,2-propanediamine, ethylenediamine and/or an alkali metal oxalate.

In particular, G1 does not comprise any silver and/or any silver compound.

In a particularly preferred embodiment of the invention, the mixture G1 comprises zinc acetate, ethylenediamine and preferably at least one solvent, with the solvent more preferably being water.

The present invention therefore also provides a process for preparing a catalyst, which comprises silver and zinc applied to a support, and a catalyst which has been or can be produced by this process, where the process comprises at least
(i) providing a support,
(ii) applying zinc to the support according to (i) in an amount of from 10 ppm to 1500 ppm, in each case calculated as element and based on the total weight of the support according to (i), by bringing the support into contact with at least one mixture G1 comprising zinc acetate and ethylenediamine,
(iii) drying and/or calcining the support obtained according to (ii),
(iv) applying silver to the dried and/or calcined support by bringing the support into contact with at least one mixture G2 comprising at least one silver compound.

The application can in principle be carried out by any suitable process, for example by impregnation of the support and optionally subsequent drying. Application is particularly preferably carried out by vacuum impregnation at room temperature. In vacuum impregnation, the support is preferably firstly treated at a pressure in the range of not more than 500 mbar, more preferably at a pressure of not more than 250 mbar and particularly preferably at a pressure of not more than 30 mbar (vacuum treatment). This is particularly preferably carried out at a temperature in the range from 1° C. to 80° C., more preferably at a temperature in the range from 3° C. to 50° C., more preferably at a temperature in the range from 5° C. to 30° C. and particularly preferably at room temperature. The vacuum treatment is preferably carried out for a time of at least 1 minute, preferably at least 5 minutes, more preferably for a time in the range from 5 minutes to 120 minutes, in particular in the range from 10 minutes to 45 minutes, particularly preferably in the range from 15 minutes to 30 minutes.

After the vacuum treatment, mixture G1 is brought into contact with the support. G1 is preferably a solution, and the solution is dripped on or sprayed on, preferably sprayed on, under reduced pressure. Application is preferably effected by means of a nozzle. The impregnation is carried out at a pressure in the range of not more than 500 mbar, more preferably at a pressure of not more than 250 mbar and particularly preferably at a pressure of not more than 30 mbar. During the impregnation, the support which has been pretreated under reduced pressure is preferably kept in motion, for example by rotating the impregnation vessel, in order to ensure very uniform distribution of the mixture G1 on the support.

As regards the amount of zinc which is applied to the support in (ii), zinc is, as mentioned above, preferably applied in an amount of from 10 to 1500 ppm, more preferably in an amount of from 50 to 1250 ppm and particularly preferably in an amount of from 150 to 1100 ppm, calculated as element and based on the total weight of the support, to the support in (ii).

The application according to (ii) can also be carried out in more than one step, for example in 2, 3 or 4 steps. Between the individual steps, the support can in each case optionally be dried and/or calcined. If the application according to (ii) is carried out in more than one step, the total amount of the zinc applied to the support after all steps is likewise in the range from 10 ppm to 1500 ppm, as described above.

Step (iii)

After the application, the support is dried and/or calcined.

Drying can in principle be carried out by any method known to those skilled in the art.

For the purposes of the invention, drying is preferably carried out at temperatures in the range from 2 to 200° C. In particular, drying is carried out by means of vacuum treatment. The term “vacuum treatment” in this context means that the support is evacuated at a pressure in the range of not more than 500 mbar, more preferably at a pressure of not more than 250 mbar and particularly preferably at a pressure of not more than 30 mbar. The vacuum treatment is preferably carried out at a temperature in the range from 2° C. to 50° C., more preferably at a temperature in the range from 5° C. to 30° C. and particularly preferably at room temperature. The vacuum treatment can be carried out at a constant temperature. Furthermore, embodiments in which the temperature is changed continuously or discontinuously during the vacuum treatment are comprised.

The vacuum treatment is carried out for a time of at least 1 minute, preferably at least 5 minutes, more preferably for a time in the range from 5 minutes to 120 minutes, in particular in the range from 10 minutes to 45 minutes, particularly preferably in the range from 10 minutes to 20 minutes.

If the support is calcined in step (iii), this calcination is preferably carried out at temperatures in the range from 150 to 1500° C., preferably in the range from 200 to 1250° C. and particularly preferably in the range from 220 to 1100° C. The calcination can be carried out in one stage or else in at least two stages. Calcination in a plurality of stages in this context means that the catalyst is cooled to a temperature in the range from 0 to 50° C., preferably to room temperature, between the respective calcination stages. The temperatures of the individual calcination stages can be identical or can differ from one another. The temperatures of the individual calcination stages preferably differ by at least 700° C. If, for example, two calcination steps are carried out in (iii), the first calcination step is preferably carried out at a temperature in the range from 150° C. to 400° C., more preferably at a temperature in the range from 200 to 350° C. and particularly preferably in the range from 220 to 300° C. The second calcination step is preferably carried out at a temperature in the range from 700° C. to 1500° C., preferably in the range from 800 to 1200° C. and particularly preferably in the range from 900 to 1100° C.

The calcination time of the at least one calcination stage is generally at least 5 minutes or more, for example in the range from 5 minutes to 24 hours or in the range from 10 minutes to 12 hours, with the calcination times of the respective stages, if a plurality of calcination stages are carried out, being able to be identical or to differ from one another.

The calcination in each stage can independently be carried out at a constant temperature; furthermore, embodiments in which the temperature is changed continuously or discontinuously during the calcination time are comprised.

The support is preferably calcined in step (iii), with the calcination particularly preferably being carried out in two calcination stages.

The calcination can be carried out under any gas atmosphere suitable for this purpose, for example in an inert gas or a mixture of an inert gas and up to 21% by volume of oxygen. Inert gases which may be mentioned are, for example, nitrogen, argon, helium, steam, carbon dioxide and combinations of the abovementioned inert gases. If the calcination is carried out in an inert gas, particular preference is given to nitrogen. In an alternative preferred embodiment, air and/or lean air is used. If the calcination is carried out in at least two calcination stages, preferably in two calcination stages, the calcination of the individual stages can be carried out in different gas atmospheres. The calcination is preferably carried out in the two stages under air and/or lean air.

The term lean air as used for the purposes of the present invention refers to air having an oxygen content in the range from 0.1 to 19% by volume and a nitrogen content of from 81 to 99.9% by volume.

Furthermore, the calcination is preferably carried out in a muffle furnace, in a rotary furnace, convection oven and/or a belt calcination furnace. If the calcination is carried out in at least two stages, preferably in two stages, the calcination is preferably carried out in different furnaces, for example firstly in a convection oven or belt calcination furnace and the second calcination step is carried out in a muffle furnace.

In step (iii), the support is particularly preferably firstly calcined at a temperature in the range from 220 to 350° C. for a time in the range from 5 minutes to 2 hours, preferably in the range from 5 minutes to 1 hour, very particularly preferably in the range from 10 minutes to 20 minutes, preferably in air, with this calcination particularly preferably being carried out in a convection oven. In a further preferred embodiment, the calcined support obtained in this way is, in step (iii), calcined in a second calcination step at a temperature in the range from 900 to 1100° C. for a time in the range from 10 minutes to 12 hours, preferably in the range from 2 hours to 11 hours, very particularly preferably in the range from 5 hours to 10 hours, preferably in air, with this calcination particularly preferably being carried out in a muffle furnace.

The present invention therefore also provides a process for preparing a catalyst, which comprises silver and zinc applied to a support, and a catalyst which has been or can be produced by this process, where the process comprises at least
(i) providing a support,
(ii) applying zinc to the support according to (i) in an amount of from 10 ppm to 1500 ppm, in each case calculated as element and based on the total weight of the support according to (i), by bringing the support into contact with at least one mixture G1 comprising at least one zinc compound,
(iii) optionally drying the support obtained according to (ii) and calcining the optionally dried support obtained according to (ii), where the calcination comprises
   (aa) calcining the optionally dried support at a temperature in the range from 220 to 350° C., preferably for a time in the range from 5 minutes to 2 hours, with the calcination preferably being carried out in air and this calcination particularly preferably being carried out in a convection oven,
   (bb) calcining the support obtained according to (aa) in a calcination step at a temperature in the range from 900 to 1100° C., preferably for a time in the range from 10 minutes to 12 hours, with the calcination preferably being carried out in air and this calcination particularly preferably being carried out in a muffle furnace,
(iv) applying silver to the optionally (IV) dried, calcined support by bringing the support into contact with at least one mixture G2 comprising at least one silver compound.

### Step (iv)

The application of silver to the support obtained according to (iii) is carried out, as described above, by bringing the support into contact with at least one mixture G2 comprising at least one silver compound.

As regards the application of silver to the support, all processes in which silver is applied in a suitable way to the support are generally suitable. Preference is given to applying the at least one mixture G2 which comprises at least one silver compound to the support by impregnation or spraying or mixing processes. Mention may be made by way of example of the preparation processes for silver catalysts as disclosed in DE-A 2300512, DE-A 2521906, EP-A 14457, EP-A 85237, EP-A 384312, DE-A 2454972, DE-A 3321895, EP-A 229465, DE-A 3150205, EP-A 172565 and EP-A 357293.

The application of silver is particularly preferably carried out by vacuum impregnation at room temperature. In vacuum impregnation, the support as described above is preferably firstly treated at a pressure in the range of not more than 500 mbar, more preferably at a pressure of not more than 250 mbar and particularly preferably at a pressure of not more than 30 mbar. This is particularly preferably carried out at a temperature in the range from 1° C. to 80° C., more preferably at a temperature in the range from 3° C. to 50° C., more preferably at a temperature in the range from 5° C. to 30° C. and particularly preferably at room temperature. The vacuum treatment is preferably carried out for a time of at least 1 minute, preferably at least 5 minutes, more preferably for a time in the range from 5 minutes to 120 minutes, in particular in the range from 10 minutes to 45 minutes, particularly preferably in the range from 15 minutes to 30 minutes.

After the vacuum treatment, at least mixture G2 is brought into contact with the support. Mixture G2 is preferably dripped on or sprayed on, preferably sprayed on. Application is preferably carried out by means of a nozzle. The impregnation is carried out at a pressure in the range of not more than 500 mbar, more preferably at a pressure of not more than 250 mbar and particularly preferably at a pressure of not more than 30 mbar. During the impregnation, the support which has been pretreated under reduced pressure is preferably kept in motion in the impregnation vessel, for example by means of rotation, in order to ensure very uniform distribution of the mixture G2 on the support.

Mixture G2 preferably comprises silver in the form of at least one silver compound. The silver compound is preferably applied as a solution, in particular as a solution in water. Accordingly, G2 also preferably comprises at least one solvent, preferably water. To obtain the silver compound in soluble form, a complexing agent such as ethanolamine, EDTA, 1,3- or 1,2-propanediamine, ethylenediamine and/or an alkali metal oxalate, which can simultaneously also act as reducing agent, can be added to the silver compound, for example silver(I) oxide or silver(I) oxalate. Silver is particularly preferably applied in the form of a silver-amine compound, in particular in the form of a silver-ethylenediamine compound.

In a preferred embodiment, G2 accordingly comprises at least one complexing agent, in particular ethanolamine, EDTA, 1,3- or 1,2-propane-diamine and/or ethylenediamine.

If G2 comprises at least one complexing agent, G2 comprises at least part of the silver in the form of a silver complex. Particular preference is given to G2 comprising at least part of the silver as silver-ethylenediamine compound. G2 particularly preferably comprises water, silver-ethylenediamine, oxalate and optionally ethylenediamine.

The present invention therefore also provides a process as described above in which G2 comprises water and silver-ethylenediamine. The present invention likewise provides a catalyst which can be or has been produced by this process.

In particular, G2 comprises neither metallic zinc nor a zinc compound.

As regards the amount of silver which is applied to the support in (iv), silver is, as mentioned above, preferably applied in an amount of from 1 to 50% by weight, more preferably in an amount of from 5 to 35% by weight and particularly preferably in an amount of from 10 to 25% by weight, calculated as element and based on the total weight of the catalyst, to the support.

The application in (iv) can also be carried out in more than one step, for example in 2, 3 or 4 steps. The support can be optionally dried and/or calcined between each of the individual steps. If the application according to (iv) is carried out in more than one step, the total amount of the silver applied to the support after all steps is likewise in the range from 1 to 50% by weight, more preferably in the range from 5 to 35% by weight and particularly preferably in an amount of from 10 to 25% by weight, calculated as element and based on the total weight of the catalyst as described above.

The application of the silver can be followed by at least one after-treatment step, for example a drying step, for example one, two or more drying steps. Drying is usually carried out at temperatures in the range from 2 to 200° C. The after-treatment step is preferably drying by means of vacuum treatment as described above. This evacuation is preferably carried out at a pressure in the range of not more than 500 mbar, more preferably at a pressure of not more than 250 mbar and particularly preferably at a pressure of not more than 30 mbar. The vacuum treatment is preferably carried out at a temperature in the range from 2° C. to 50° C., more preferably at a temperature in the range from 5° C. to 30° C. and particularly preferably at room temperature. The vacuum treatment is carried out for a time of at least 1 minute, preferably at least 5 minutes, more preferably for a time in the range from 5 minutes to 120 minutes, in particular in the range from 10 minutes to 45 minutes, particularly preferably in the range from 10 minutes to 20 minutes.

Application of the silver and optionally the at least one drying step is/are preferably followed by at least one calcination step.

The present invention therefore also provides a process as described above and a catalyst which can be or has been produced by this process, where the process further comprises
(v) drying and/or calcining the support according to (iv).

If a calcination is carried out in step (v), this calcination is preferably carried out at temperatures in the range from 150 to 750° C., preferably in the range from 200 to 500° C. and particularly preferably in the range from 220 to 350° C., with the calcination time generally being at least 5 minutes or more, for example in the range from 5 minutes to 24 hours or in the range from 10 minutes to 12 hours.

The calcination time is particularly preferably in the range from 5 minutes to 3 hours. The calcination can be carried out at a constant temperature; furthermore, embodiments in which the temperature is changed continuously or discontinuously during the calcination time are comprised.

The calcination can, as described above, be carried out under any gas atmosphere suitable for this purpose, for example in an inert gas or a mixture of an inert gas and up to 21% by volume of oxygen. As inert gases, mention may be made by way of example of nitrogen, argon, helium, carbon dioxide, steam and combinations of the abovementioned inert gases. If the calcination is carried out in an inert gas, particular preference is given to nitrogen. In an alternative preferred embodiment, air and/or lean air is/are used. Furthermore, the calcination is preferably carried out in a muffle furnace, in a rotary furnace, convection oven and/or a belt calcination furnace.

### Promoters

The catalyst preferably comprises at least one promoter in addition to silver and zinc. This at least one promoter is preferably applied to the support by impregnation or spraying or mixing processes.

As regards the point in time at which the at least one promoter is applied, this can be applied after the above-described calcination according to step (v). As an alternative, it is possible to apply the at least one promoter together with the silver compound to the support.

Accordingly, the invention comprises embodiments in which the at least one promoter, i.e., for example, five different promoters, four different promoters, three different promoters, two different promoters or one promoter, are applied to the support and the support which has been treated in this way is only then calcined as described above to give a catalyst according to the invention.

Embodiments in which the promoter is applied in step (ii), before step (ii) or after step (ii) and/or (iii) are likewise comprised. It is likewise possible to apply the at least one promoter after step (iv).

Furthermore, embodiments in which part of the at least one promoter is applied to the support only after application of the silver according to (iv) and the remaining part is applied simultaneously with silver in (iv).

For the purposes of the invention, the at least one promoter is preferably applied simultaneously with silver in step (iv). Here, the at least one promoter can be applied in a further solution G2′ to the support. The promoter is preferably applied together with silver. In this case, G2 further comprises the at least one promoter.

If the catalyst comprises at least one promoter, this at least one promoter is preferably applied in the form of compounds to the support, for example in the form of complexes or in the form of salts, for example in the form of halides, for example in the form of fluorides, bromides or chlorides, or in the form of carboxylates, nitrates, sulfates or sulfides, phosphates, cyanides, hydroxides, carbonates or as salts or heteropolyacids, in the process of the invention for preparing the catalyst.

### Rhenium as Promoter

The catalyst particularly preferably comprises rhenium as promoter. Rhenium is preferably applied together with silver in step (iv) of the process of the invention. The rhenium is particularly applied as a compound, for example as halide, oxyhalide, oxide or as acid. Furthermore, rhenium can be used in the form of salts of heteropolyacids of rhenium, for example, as rhenate or perrhenate, in the preparation process of the invention.

The present invention therefore also provides a process as described above and a catalyst which can be or has been produced by this process, where the catalyst comprises rhenium as promoter and the rhenium is applied to the dried and/or calcined support according to (iii) by impregnation with the mixture G2 which additionally comprises at least one rhenium compound in step (iv).

If rhenium is to be used as promoter, it is preferably applied as a compound selected from the group consisting of ammonium perrhenate, rhenium(III) chloride, rhenium(V) chloride, rhenium(V) fluoride, rhenium(VI) oxide and rhenium(VII) oxide. For the purposes of the invention, rhenium is particularly preferably applied as ammonium perrhenate to the support.

As regards the amount of rhenium, the catalyst preferably comprises rhenium in an amount of from 50 ppm to 1000 ppm, more preferably in an amount of from 100 ppm to 800 ppm, more preferably in an amount of from 200 ppm to 600 ppm and particularly preferably in an amount of from 250 ppm to 450 ppm, based on the total weight of the catalyst and calculated as element.

The present invention therefore also provides a process as described above and a catalyst which can be or has been produced by this process, wherein the catalyst comprises rhenium as promoter, preferably in an amount of from 50 ppm to 1000 ppm, based on the total weight of the catalyst and calculated as element. The present invention likewise describes a catalyst itself as described above, wherein the catalyst comprises rhenium as promoter, preferably in an amount of from 50 ppm to 1000 ppm, based on the total weight of the catalyst and calculated as element.

### Further Promoters

In a particularly preferred embodiment, the catalyst additionally comprises at least one further promoter. This further promoter is particularly selected from among elements of groups IA, VIB, VIIB and VIA of the Periodic Table of the Elements, particularly preferably selected from the group consisting of tungsten, lithium, sulfur, cesium, chromium, manganese, molybdenum and potassium.

The present invention therefore also provides a process as described above and a catalyst which can be or has been produced by this process, wherein the catalyst additionally comprises at least one further promoter selected from among elements of groups IA, VIIB, VIIB and VIA of the Periodic Table of the Elements, preferably selected from the group consisting of tungsten, lithium, sulfur, cesium, chromium, manganese, molybdenum and potassium.

In a particularly preferred embodiment, the catalyst additionally comprises cesium, lithium, tungsten and sulfur as promoters.

If the catalyst comprises at least one further promoter, it preferably comprises a total amount of these further promoters of from 10 to 2000 ppm, preferably from 10 to 1700 ppm, more preferably in each case from 50 to 1500 ppm and particularly preferably in each case from 80 to 1200 ppm, based on the total weight of the catalyst and calculated as sum of the elements.

If the catalyst comprises tungsten as promoter as described above, the tungsten is preferably applied as tungsten compound to the support. Here, it is in principle possible to use any suitable tungsten compound. For example, tungsten is applied in the form of tungstate or tungstic acid.

If the catalyst comprises lithium as promoter as described above, the lithium is preferably applied as lithium compound to the support. Here, it is in principle possible to use any suitable lithium compound. Lithium is preferably applied in the form of lithium nitrate.

If the catalyst comprises cesium as promoter as described above, the cesium is preferably applied as cesium compound to the support. Here, it is in principle possible to use any suitable cesium compound. Cesium is preferably applied in the form of cesium hydroxide.

If the catalyst comprises sulfur as promoter as described above, the sulfur is preferably applied as sulfur compound to the support. Here, it is in principle possible to use any suitable sulfur compound. Sulfur is preferably applied in the form of ammonium sulfate.

The at least one further promoter, more preferably the at least one further promoter compound, is preferably dissolved in a suitable solution, preferably water, before application. The support is then preferably impregnated with the resulting solution comprising one or more of the further promoters. If a plurality of further promoters are to be added, these can be applied either together or separately to the support in a single impregnation step or in a plurality of impregnation steps. As regards the solution comprising one or more of the further promoters, this can be produced in any suitable way. For example, the promoters can each be dissolved separately in a solution and the resulting solutions, each comprising a promoter, can subsequently be used for the impregnation. It is likewise possible for two or more of the further promoters to be dissolved together in a solution and the resulting solution subsequently to be used for the impregnation. In addition, it is possible to combine the resulting solutions comprising at least one promoter before impregnation and apply the resulting solution comprising all promoters to the support.

If, for example, at least rhenium as promoter and cesium, tungsten, lithium, sulfur as further promoters are used, at least one solution comprising cesium and tungsten, a further solution comprising lithium and sulfur and a further solution comprising rhenium are produced in a particularly preferred embodiment. The solutions are either applied to the support in separate impregnation steps or are combined to form one solution before application and only then used for the impregnation. The solutions or the combined solution is/are preferably applied together with G2.

The solutions are particularly preferably combined with a solution comprising at least one silver compound to give the mixture G2. Thus, G2 particularly preferably comprises the at least one silver compound, at least one rhenium compound, at least one cesium compound, at least one lithium compound, at least one tungsten compound and optionally further promoters, in each case in the form of a compound.

The present invention therefore also provides a process as described above and a catalyst which can be produced by this process, wherein the catalyst additionally comprises at least one further promoter selected from among elements of groups IA, VIB, VIIB and VIA of the Periodic Table of the Elements, preferably selected from the group consisting of tungsten, lithium, sulfur, cesium, chromium, manganese, molybdenum and potassium, and the at least one further promoter is preferably applied to the dried and/or calcined support by impregnation with the mixture G2 which additionally comprises the at least one promoter in step (iv).

In a particularly preferred embodiment, the catalyst comprises rhenium in an amount of from 150 to 450 ppm, tungsten in an amount of from 10 to 300 ppm, cesium in an amount of from 50 to 700 ppm, lithium in an amount of from 50 to 300 ppm and sulfur in an amount of from 5 to 150 ppm.

The present invention therefore also provides a process as described above and a catalyst which can be produced by this process, where the catalyst comprises rhenium as promoter in an amount of from 150 to 450 ppm and as further promoters at least tungsten in an amount of from 10 to 300 ppm, cesium in an amount of from 50 to 700 ppm, lithium in an amount of from 50 to 300 ppm and sulfur in an amount of from 5 to 150 ppm.

### Process for Preparing Ethylene Oxide

The catalysts of the invention and the catalysts which can be obtained by a process according to the invention are particularly suitable as catalysts for preparing ethylene oxide from ethylene, which comprises an oxidation of ethylene. High selectivities, in particular advantageous initial selectivities, and good activities are achieved.

The present invention therefore also provides, according to a further aspect, a process for preparing ethylene oxide from ethylene, which comprises oxidation of ethylene in the presence of a catalyst for preparing ethylene oxide, as described above.

In addition, the present invention also provides for the use of a catalyst as described above for the preparation of ethylene oxide.

According to the invention, the epoxidation can be carried out by all processes known to those skilled in the art. Here, it is possible to use all reactors which can be used in the ethylene oxide preparation processes of the prior art, for example externally cooled shell-and-tube reactors (cf. Ullmann's Encyclopedia of Industrial Chemistry, 5^{th} edition, vol. A-10, pp. 117-135, 123-125, VCH Verlagsgesellschaft, Weinheim 1987) or reactors having a loose catalyst bed and cooling tubes, for example the reactors described in DE-A 3414717, EP-A 0 082 609 and EP-A 0 339 748. The epoxidation is preferably carried out in at least one tube reactor, preferably in a shell-and-tube reactor.

To prepare ethylene oxide from ethylene and oxygen, it is possible according to the invention to work under conventional reaction conditions as described, for example, in DE 25 21 906 A1, EP 0 014 457 A2, DE 2 300 512 A1, EP 0 172 565 A2, DE 24 54 972 A1, EP 0 357 293 A1, EP 0 266 015 A1, EP 0 085 237 A1, EP 0 082 609 A1 and EP 0 339 748 A2. Inert gases such as nitrogen or gases which are inert under the reaction conditions, e.g. steam, methane, and also optionally reaction moderators, for example halides, hydrocarbons such as ethyl chloride, vinyl chloride or 1,2-dichloroethane, can be additionally mixed into the reaction gas comprising ethylene and molecular oxygen. The oxygen content of the reaction gas is advantageously in a range in which no explosive gas mixtures are present. A suitable composition of the reaction gas for preparing ethylene oxide can comprise, for example, an amount of ethylene in the range from 10 to 80% by volume, preferably from 20 to 60% by volume, more preferably from 25 to 50% by volume and particularly preferably in the range from 30 to 40% by volume, based on the total volume of the reaction gas. The oxygen content of the reaction gas is advantageously in the range of not more than 10% by volume, preferably not more than 9% by volume, more preferably not more than 8% by volume and very particularly preferably not more than 7% by volume, based on the total volume of the reaction gas.

The reaction gas preferably comprises a chlorine-comprising reaction moderator such as ethyl chloride, vinyl chloride or dichloroethane in an amount of from 0 to 15 ppm, preferably in an amount of from 0.1 to 8 ppm. The remainder of the reaction gas generally comprises hydrocarbons such as methane or else inert gases such as nitrogen. In addition, further materials such as steam, carbon dioxide or noble gases can also be comprised in the reaction gas.

The above-described constituents of the reaction mixture can optionally each have small amounts of impurities. Ethylene can, for example, be used in any purity suitable for the gas-phase oxidation according to the invention. Suitable purities include but are not limited to polymer-grade ethylene which typically has a purity of at least 99% and chemical-grade ethylene which has a low purity of typically less than 95%. The impurities typically comprise, in particular, ethane, propane and/or propene.

The reaction or oxidation of ethylene to ethylene oxide is usually carried out at elevated temperature. Preference is given to temperatures in the range from 150 to 350° C., more preferably in the range from 180 to 300° C., more preferably in the range from 190° C. to 280° C. and particularly preferably in the range from 200° C. to 280° C. The present invention therefore also provides a process as described above in which the oxidation is carried out at a temperature in the range from 180 to 300° C., preferably in the range from 200 to 280° C.

The reaction (oxidation) according to the invention is preferably carried out at pressures in the range from 5 bar to 30 bar. The oxidation is more preferably carried out at a pressure in the range from 5 bar to 25 bar, preferably at a pressure in the range from 10 bar to 20 bar and in particular in the range from 14 bar to 20 bar. The present invention therefore also provides a process as described above in which the oxidation is carried out at a pressure in the range from 14 bar to 20 bar.

The oxidation is preferably carried out in a continuous process. If the reaction is carried out continuously, a GHSV (gas hourly space velocity) which is, depending on the type of reactor selected, for example on the size/cross-sectional area of the reactor, the shape and size of the catalyst, preferably in the range from 800 to 10000/h, preferably in the range from 2000 to 6000/h, more preferably in the range from 2500 to 5000/h is used, where the values indicated are based on the volume of the catalyst.

The preparation of ethylene oxide from ethylene and oxygen can advantageously be carried out in a recycled process. Here, the reaction mixture is circulated through the reactor, with the newly formed ethylene oxide and also the by-products formed in the reaction being removed from the product gas stream after each pass and the remainder being, after being supplemented with the required amount of ethylene, oxygen and reaction moderators, recirculated to the reactor. The separation of the ethylene oxide from the product gas stream and the work-up thereof can be carried out by the customary processes of the prior art (cf. Ullmann's Encyclopedia of Industrial Chemistry, 5^{th} edition, vol. A-10, pp. 117-135, 123-125, VCH Verlagsgesellschaft, Weinheim 1987).

Particularly preferred embodiments of the invention are indicated below, including the embodiments resulting from the combinations given by the back-references being explicitly:
1. A process for preparing a catalyst for preparing ethylene oxide, which comprises silver and zinc applied to a support, where the process comprises at least
   (i) providing the support,
   (ii) applying zinc to the support according to (i) in an amount of from 10 ppm to 1500 ppm, in each case calculated as element and based on the total weight of the support according to (i), by bringing the support into contact with at least one mixture G1 comprising at least one zinc compound,
   (iii) drying and/or calcining the support obtained according to (ii),
   (iv) applying silver to the dried and/or calcined support by bringing the support into contact with at least one mixture G2 comprising at least one silver compound.
2. The process according to embodiment 1, wherein the catalyst comprises at least rhenium as promoter, preferably in an amount of from 50 ppm to 1000 ppm, based on the total weight of the catalyst and calculated as element.
3. The process according to embodiment 2, wherein the rhenium is applied to the dried and/or calcined support according to (iii) by impregnation with the mixture G2 which additionally comprises at least one rhenium compound in step (iv).
4. The process according to any of embodiments 1 to 3, wherein the support is an alumina support, preferably an alpha-alumina support.
5. The process according to embodiment 4, wherein the alumina support is an alpha-alumina support having a purity, based on alpha-alumina, of at least 85%.
6. The process according to embodiment 5, wherein the alumina support comprises calcium in an amount in the range from 10 to 1500 ppm, based on the total weight of the support and calculated as element.
7. The process according to embodiment 5 or 6, wherein the alumina support comprises magnesium in an amount in the range of not more than 800 ppm, preferably in an amount of from 1 to 500 ppm, based on the total weight of the support and calculated as element.
8. The process according to any of embodiments 5 to 7, wherein the alumina support comprises potassium in an amount in the range of not more than 1000 ppm, based on the total weight of the support and calculated as element.
9. The process according to any of embodiments 5 to 8, wherein the alumina support comprises sodium in an amount in the range from 10 to 1500 ppm, based on the total weight of the support and calculated as element.
10. The process according to any of embodiments 5 to 9, wherein the alumina support comprises silicon in an amount in the range from 50 to 10000 ppm, more preferably in an amount of from 100 to 5000 ppm, more preferably in an amount of from 1000 to 2500 ppm, based on the total weight of the support and calculated as element.
11. The process according to any of embodiments 5 to 10, wherein the alumina support comprises zirconium in an amount in the range from 1 to 10000 ppm, based on the total weight of the support and calculated as element.
12. The process according to any of embodiments 1 to 11, wherein the support has a BET surface area determined in accordance with DIN ISO 9277 in the range from 0.5 to 1.5 m²/g.
13. The process according to any of embodiments 1 to 12, wherein the support has a bimodal pore distribution, preferably a bimodal pore distribution at least comprising pores having pore diameters in the range from 0.1 to 10 μM and pores having pore diameters in the range from 15 to 100 μm, determined by Hg porosimetry in accordance with DIN 66133.
14. The process according to any of embodiments 1 to 13, wherein mixture G1 comprises zinc acetate and ethylenediamine.
15. The process according to any of embodiments 1 to 14, wherein zinc is applied in an amount of from 50 to 1250 ppm, calculated as element and based on the total weight of the support according to (i), to the support in (ii).
16. The process according to any of embodiments 1 to 15, wherein the support provided according to (i) additionally comprises from 1 to 1000 ppm of zinc and from 1 to 10000 ppm of zirconium, in each case calculated as element and based on the total weight of the support according to (i).
17. The process according to any of embodiments 1 to 16, wherein the catalyst additionally comprises at least one further promoter selected from among elements of groups IA, VIB, VIIB and VIA of the Periodic Table of the Elements, preferably selected from the group consisting of tungsten, lithium, sulfur, cesium, chromium, manganese, molybdenum and potassium, where the at least one promoter is preferably applied to the dried and/or calcined support according to (iii) by impregnation with the mixture G2 which additionally comprises the at least one promoter in step (iv).
18. The process according to any of embodiments 1 to 17, wherein the catalyst comprises rhenium in an amount of from 150 to 450 ppm, tungsten in an amount of from 10 to 300 ppm, cesium in an amount of from 50 to 700 ppm, lithium in an amount of from 50 to 300 ppm and sulfur in an amount of from 5 to 150 ppm.
19. The process according to any of embodiments 1 to 18, wherein the catalyst comprises silver in an amount of from 5 to 35% by weight, calculated as element and based on the total weight of the catalyst.
20. The process according to any of embodiments 1 to 19, which further comprises
   (v) drying and/or calcining the support according to (iv).
21. A catalyst for preparing ethylene oxide, which comprises at least silver and zinc applied to a support, obtainable or obtained by a process according to any of embodiments 1 to 20.
22. A process for preparing ethylene oxide from ethylene, which comprises oxidation of ethylene in the presence of a catalyst according to embodiment 21.
23. The use of a catalyst according to embodiment 21 as catalyst for the preparation of ethylene oxide from ethylene by oxidation of ethylene.

The present invention is illustrated below with the aid of examples.

### EXAMPLES

### 1. General Method for the Preparation of Catalysts According to the Invention

### 1.1 Supports Used

A bimodal alpha-alumina support having a hollow ring geometry (6.10 mm×6.31 mm×2.87 mm), comprising 400 ppm of Ca, 60 ppm of Na, 2200 ppm of Si, 5000 ppm of zirconium, 200 ppm of zinc and less than 50 ppm of iron, magnesium, potassium and having a BET surface area of 0.89 m²/g and a cold water uptake under reduced pressure of 0.47 ml/g was used.

### 1.2 Preparation and Application of the Zinc Complex Solution to the Support

The support was impregnated with a zinc complex solution comprising zinc acetate and ethylenediamine dissolved in water. For this purpose, 180.2 g of ethylenediamine were firstly placed together with 180.2 g of water in a 500 ml stirred apparatus without inert gas flushing and brought by means of ice water cooling to 10° C. 250.1 g of zinc acetate dihydrate were subsequently added slowly and stirred for a further 2 hours while cooling. This gave 611.78 g of a zinc complex solution having a proportion by mass of 12.2% by weight of zinc.

Impregnation was carried out by means of vacuum impregnation. For this purpose, the alpha-alumina support was firstly evacuated at a pressure of 6 mbar and a temperature of 22° C. in a rotary evaporator and the zinc complex solution was subsequently added dropwise. The impregnated support was subsequently allowed to rotate under reduced pressure for a further 15 minutes. The support was then left in the apparatus at room temperature and atmospheric pressure for 1 hour and gently mixed every 15 minutes.

After impregnation with zinc, the support was calcined at 280° C. for about 12 minutes in air in order to decompose the organic components of the complex solutions and deposit the metal on the support. After cooling to room temperature, the support which had been pretreated with zinc in this way was transferred to a muffle furnace and heated to 1000° C. over a period of 8 hours and then calcined at 1000° C. for a time of 2 hours. After the 2 hours had elapsed, the support was cooled to room temperature in the furnace.

### 1.3 Preparation of the Silver Complex Solution

1.5 l of water were placed in a vessel and 550 g of silver nitrate were added while stirring and completely dissolved therein. The solution was heated to 40° C. during this time. 402.62 g of potassium hydroxide solution (47.8%) were mixed with 1.29 l of water. 216.31 g of oxalic acid were subsequently added and completely dissolved and the solution was heated to 40° C. The potassium oxalate solution was subsequently added by means of a metering pump to the silver nitrate solution (40° C.) over a period of about 45 minutes (volume flow=about 33 ml/min). After the addition was complete, the resulting solution was stirred further at 40° C. for 1 hour. The precipitated silver oxalate was filtered off and the filtercake obtained was washed with 1 l portions of water (about 10 l) until it was free of potassium and nitrate (determined by means of conductivity measurement on the washings; for the present purposes, free of potassium and nitrate means a conductivity of <40 μS/cm). The water was removed as completely as possible from the filtercake and the residual moisture of the filtercake was determined. 620 g of silver oxalate having a water content of 20.80% were obtained.

309.7 g of ethylenediamine were cooled by means of an ice bath to about 10° C. and 247.9 g of water were added in small portions. After the addition of water was complete, the 620 g of the moist silver oxalate obtained (corresponding to 491.0 g of dry silver oxalate) were added in small portions over a period of about 30 minutes. The mixture was stirred overnight at RT and the residue was subsequently centrifuged off. The Ag content of the remaining clear solution was determined refractometrically and the density was determined by means of a 10 ml measuring cylinder.

The solution obtained comprised 29.35% by weight of silver, calculated as element, and had a density of 1.536 g/ml.

### 1.4 General Method for Preparing the Solution Comprising Silver and Promoters

The silver oxalate solution produced as described in method 1.3 was placed in a vessel. An aqueous solution of lithium and sulfur (lithium nitrate and (NH₄)₂SO₄), an aqueous solution of tungsten and cesium (H₂WO₄ and CsOH) and an aqueous solution of rhenium (ammonium perrhenate) were added thereto and the solution was stirred for 5 minutes.

### 1.5 Application of the Solution to the Support

120 g of the zinc-impregnated support according to general method 1.2 (see table 1) were placed in a rotary evaporator and evacuated at 6 mbar. The support was preevacuated for about 10 minutes.

The solution obtained according to general method 1.4 was dripped onto the support over a period of 15 minutes and the impregnated support was subsequently allowed to rotate under reduced pressure for a further 15 minutes. The support was then left in the apparatus at room temperature and atmospheric pressure for 1 hour and gently mixed every 15 minutes.

### 1.6 Calcination of the Impregnated Support

The impregnated support was treated at 283° C. under 8.3 m³ of air per hour in a convection oven (HORO, model 129 ALV-SP, catalogue No.: 53270) for 12 minutes.

The amount of silver and the promoters is identical for all catalysts produced.

### 1.7 Epoxidation

The epoxidation was carried out in an experimental reactor comprising an upright stainless steel reaction tube having an internal diameter of 6 mm and a length of 2200 mm. The reaction tube was provided with a jacket and was heated by means of hot oil having a temperature of T which flowed through the jacket. To a very good approximation, the temperature of the oil corresponds to the temperature in the reaction tube and thus the reaction temperature. The reaction tube was filled from the bottom upward to a height of 212 mm with inert steatite spheres (1.0-1.6 mm), then to a height of 1100 mm with 38.2 g of crushed catalyst, particle size 0.5-0.9 mm, and then to a height of 707 mm with inert steatite spheres (1.0-1.6 mm). The feed gas entered the reactor from the top and left it again at the lower end after passing through the catalyst bed.

The feed gas comprised 35% by volume of ethylene, 7% by volume of oxygen and 1% by volume of CO₂ (EC (ethylene chloride) moderation). At the beginning, 2.5 ppm of EC were used for start-up. Depending on the catalyst and performance, the EC concentration was increased every 24 h to a maximum of 8 ppm. The remainder of the feed gas comprised methane. The experiments were carried out at a pressure of 15 bar and a gas hourly space velocity (GHSV) of 4750/h and a space-time yield of 250 kg of EO/(m³(cat)×h).

The reaction temperature was regulated according to the given ethylene oxide offgas concentration of 2.7%. To optimize the catalyst in respect of selectivity and conversion, from 2.2 to 8.0 ppm of ethylene chloride were added as moderator to the feed gas.

The gas leaving the reactor was analyzed by means of on-line MS. The selectivity was determined from the analytical results.

### Example 1

170 g of the support according to 1.1 were impregnated according to method 1.2 with 1.6560 g of the zinc complex solution (12.2% by weight of Zn) and 78.26 g of water. Before preparation of the catalyst and addition of the solution according to 1.4, the water uptake of the support was redetermined. This new water uptake was 0.471 ml/g and served to calculate the amounts required for preparation of the solution according to 1.4.

The following amounts were used for preparing the solution according to general method 1.4:

89.9527 g of silver oxalate solution (28.62% of Ag),
1.1127 g of a solution comprising 2.85% of lithium and 0.21% of sulfur,
3.3228 g of a solution comprising 1.00% of tungsten and 1.75% of cesium,
1.6630 g of a solution comprising 3.1% of rhenium,
0.7699 g of water.

The support obtained according to general method 1.2 was subsequently converted into the catalyst according to methods 1.5 and 1.6. The catalyst obtained, which comprised 1000 ppm of zinc applied to the support, calculated as element and based on the total weight of the support, was subsequently tested to general method 1.7. The result is shown in table 1.

### Example 2

170 g of the support according to 1.1 were impregnated according to method 1.2 with 0.7019 g of the zinc complex solution (12.2% by weight of Zn) and 79.29 g of water. Before preparation of the catalyst and addition of the solution according to 1.4, the water uptake of the support was redetermined. This new water uptake was 0.458 ml/g and served to calculate the amounts.

The following amounts were used for preparing the solution according to general method 1.4:

88.8843 g of silver oxalate solution (28.93% of Ag),
1.1062 g of a solution comprising 2.85% of lithium and 0.21% of sulfur,
3.3236 g of a solution comprising 1.00% of tungsten and 1.75% of cesium,
1.2561 g of a solution comprising 4.1% of rhenium,
0.2401 g of water.

The support obtained according to general method 1.2 was subsequently converted into the catalyst according to methods 1.5 and 1.6. The catalyst obtained, which comprised 500 ppm of zinc applied to the support prior to the impregnation with silver, calculated as element and based on the total weight of the support, was subsequently tested according to general method 1.7. The result is shown in table 1.

### Example 3

170 g of the support according to 1.1 were impregnated according to method 1.2 with 0.4156 g of the zinc complex solution (12.2% by weight of Zn) and 79.50 g of water. Before preparation of the catalyst and addition of the solution according to 1.4, the water uptake of the support was redetermined. This new water uptake was 0.471 ml/g and served to calculate the amounts.

The following amounts were used for preparing the solution according to general method 1.4:

89.9042 g of silver oxalate solution (28.62% of Ag),
1.1233 g of a solution comprising 2.85% of lithium and 0.21% of sulfur,
3.3230 g of a solution comprising 1.00% of tungsten and 1.75% of cesium,
1.6600 g of a solution comprising 4.1% of rhenium,
0.8947 g of water.

The support obtained according to general method 1.2 was subsequently converted into the catalyst according to methods 1.5 and 1.6. The catalyst obtained, which comprised 250 ppm of zinc applied to the support prior to the impregnation with silver, calculated as element and based on the total weight of the support, was subsequently tested according to general method 1.7. The result is shown in table 1.

### Example 4

### Not According to the Invention

140 g of the support according to 1.1 were impregnated according to method 1.2 with 72.2723 g of the zinc complex solution (12.2% by weight of Zn) and 6.4738 g of water. Before preparation of the catalyst and addition of the solution according to 1.4, the water uptake of the support was redetermined. This new water uptake was 0.435 ml/g and served to calculate the amounts.

The following amounts were used for preparing the solution according to general method 1.4:

74.6531 g of silver oxalate solution (29.35% of Ag),
1.0018 g of an aqueous solution of lithium and sulfur (2.85% of lithium from lithium nitrate and 0.21% of sulfur from (NH₄)₂SO₄),
1.4966 g of an aqueous solution of tungsten and cesium (2.00% of tungsten from H₂WO₄ and 3.50% of cesium from CsOH),
1.1365 g of an aqueous solution of rhenium (4.1% of rhenium from ammonium perrhenate).

The support obtained according to general method 1.2 was subsequently converted into the catalyst according to methods 1.5 and 1.6. The catalyst obtained, which comprised 5% by weight of zinc applied to the support prior to the impregnation with silver, calculated as element and based on the total weight of the support, was subsequently tested according to general method 1.7. The result is shown in table 1.

### Example 5

### Not According to the Invention

The catalyst was produced according to general methods 1.5 to 1.6, with 0.6835 g of the zinc complex solution (12.2% by weight of Zn) and 0.6408 g of water being additionally added to the impregnation solution as described in 1.4. 140 g of support having a water uptake of 0.47 ml/g were used.

A preceding impregnation and calcination with zinc according to general methods 1.2 to 1.3 was not carried out.

Apart from 0.6835 g of the zinc complex solution (12.2% by weight of Zn) and 0.6408 g of water, the following amounts were used for preparing the solution according to general method 1.4:

88.9344 g of silver oxalate solution (28.93% of Ag),
1.1113 g of a solution comprising 2.85% of lithium and 0.21% of sulfur,
3.3216 g of a solution comprising 1.00% of tungsten and 1.75% of cesium,
1.6631 g of a solution comprising 3.1% of rhenium.

The catalyst obtained, which comprised 500 ppm of zinc together with silver and the other promoters applied to the support, calculated as element and based on the total weight of the support, was subsequently tested according to general method 1.7. The result is shown in table 1.

### Example 6

### Not According to the Invention

The catalyst was produced according to general methods 1.5 to 1.6. Impregnation with zinc according to general methods 1.2 to 1.3 was not carried out.

The following amounts were used for preparing the solution according to general method 1.4:

59.4199 g of silver oxalate solution (29.04% of Ag),
0.7841 g of a solution comprising 2.85% of lithium and 0.21% of sulfur,
1.1751 g of a solution comprising 2.00% of tungsten and 3.50% of cesium,
1.1749 g of a solution comprising 3.1% of rhenium, 4.9638 g of water.

100 g of support having a water uptake of 0.47 ml/g according to methods 1.5 and 1.6 were treated with this solution. The catalyst obtained was subsequently tested according to general method 1.7. The result is shown in table 1 and FIG. 1.

### Results

Composition of the catalysts as per examples 1 to 6: the catalysts comprise 14.8% of Ag, 190 ppm of Li, 14 ppm of S, 200 ppm of W, 350 ppm of Cs, 310 ppm of Re and various amounts of zinc as indicated.

It was able to be shown that impregnation of the support with zinc complex solution and subsequent calcination before impregnation with silver has a positive effect on the initial selectivity. Compared to the catalyst which has not been preimpregnated with zinc, the addition of zinc leads to a better “start of run” selectivity of up to 3.3%, with otherwise virtually identical catalytic behavior. When the zinc concentration is too high or zinc is applied simultaneously with the other constituents of the catalyst, the selectivity of the catalyst decreases significantly (examples 4 and 5).

TABLE 1
Catalytic results for examples 1 to 6

| | | Start of run | Temperature/ |
|---|---|---|---|
| | Zinc source | selectivity/% | ° C. |
| Example 1 | Complex solution 1000 ppm | 88.4% | 237 |
| Example 2 | Complex solution 500 ppm | 88.1% | 239 |
| Example 3 | Complex solution 250 ppm | 87.5% | 237 |
| Example 4 | Complex solution 5% | 79.5% | 229 |
| Example 5 | Complex solution 500 ppm | 82.9% | 236 |
| Example 6 | — | 85.1% | 238 |

1. A process for preparing a catalyst for preparing ethylene oxide, which comprises silver and zinc applied to a support, where the process comprises at least
(i) providing the support,
(ii) applying zinc to the support according to (i) in an amount of from 10 ppm to 1500 ppm, in each case calculated as element and based on the total weight of the support according to (i), by bringing the support into contact with at least one mixture G1 comprising at least one zinc compound,
(iii) drying and/or calcining the support obtained according to (ii),
(iv) applying silver to the dried and/or calcined support according to (iii) by bringing the support into contact with at least one mixture G2 comprising at least one silver compound.

2. The process according to claim 1, wherein the catalyst comprises at least rhenium as promoter.

3. The process according to claim 2, wherein the rhenium is applied to the dried and/or calcined support according to (iii) by impregnation with the mixture G2 which additionally comprises at least one rhenium compound in step (iv).

4. The process according to claim 1, wherein the support is an alumina support.

5. The process according to claim 1, wherein the support has a BET surface area determined in accordance with DIN ISO 9277 in the range from 0.5 to 1.5 m²/g.

6. The process according to claim 1, wherein the support has a bimodal pore distribution.

7. The process according to claim 1, wherein the mixture G1 comprises zinc acetate and ethylenediamine.

8. The process according to claim 1, wherein zinc is applied in an amount of from 50 to 1250 ppm, calculated as element and based on the total weight of the support according to (i), to the support in (ii).

9. The process according to claim 1, wherein the support provided according to (i) additionally comprises from 1 to 1000 ppm of zinc and from 1 to 10000 ppm of zirconium in each case calculated as element and based on the total weight of the support according to (i).

10. The process according to claim 1, wherein the catalyst additionally comprises at least one further promoter selected from among elements of groups IA, VIB, VIIB and VIA of the Periodic Table of the Elements, where the at least one promoter is applied to the dried and/or calcined support according to (iii) by impregnation with the mixture G2 which additionally comprises the at least one promoter in step (iv).

11. The process according to claim 1, wherein the catalyst comprises silver in an amount of from 5 to 35% by weight, calculated as element and based on the total weight of the catalyst.

12. The process according to claim 1, which further comprises
(v) drying and/or calcining the support obtained according to (iv).

13. A catalyst for preparing ethylene oxide, which comprises at least silver and zinc applied to a support, obtainable or obtained by a process according to claim 1.

14. A process for preparing ethylene oxide from ethylene, which comprises oxidation of ethylene in the presence of a catalyst according to claim 13.

15. (canceled)

16. The process according to claim 1, wherein the catalyst comprises at least rhenium as promoter, in an amount of from 50 ppm to 1000 ppm, based on the total weight of the catalyst and calculated as element.

17. The process according to claim 1, wherein the support is an alpha-alumina support.

18. The process according to claim 1, wherein the support has a bimodal pore distribution at least comprising pores having pore diameters in the range from 0.1 to 10 μm and pores having pore diameters in the range from 15 to 100 μm, determined by Hg porosimetry in accordance with DIN 66133.

19. The process according to claim 1, wherein the catalyst additionally comprises at least one further promoter selected from the group consisting of tungsten, lithium, sulfur, cesium, chromium, manganese, molybdenum and potassium, where the at least one promoter is applied to the dried and/or calcined support according to (iii) by impregnation with the mixture G2 which additionally comprises the at least one promoter in step (iv).
